(19) United States
(12) Patent Application Publication
(10) Pub. No.: US 2006/0105940 A1
(43) Pub. Date: May 18, 2006

(54) Compound useful in the treatment or prevention of cognitive disorders associated with diabetes and associated methods

(75) Inventors: Nigel H. Greig, Phoenix, MD (US); Gosse B. Bruinsma, Leiden (NL); Qian-Sheng Yu, Lutherville, MD (US)

Correspondence Address: TRASK BRITT, P.O. BOX 2550, SALT LAKE CITY, UT, 84110, US

(73) Assignee: Axonyx, Inc., New York, NY (US)

(21) Appl. No.: 11/261,121

(22) Filed: Oct. 28, 2005

Related U.S. Application Data
(60) Provisional application No. 60/624,717, filed on Nov. 3, 2004.

(51) Int. Cl. A61K 31/4439; A61K 31/426; A61K 31/407; A61K 38/28; A61K 31/155; A61K 31/175

(52) U.S. Cl. 514/3; 514/340; 514/369; 514/411; 514/592; 514/635

(57) ABSTRACT

Described is the efficient synthesis of an easy to manipulate and utilize, soluble tartrate salt of a potent, reversible butyrylcholinesterase inhibitor, (−)-(3aS)-3a-methyl-1,2,3,3a,8,8a-hexahydropyrrolo-[2,3-b]indol-5-yl N—4′-isopropylphenylcarbamate (“MHI tartrate”), for use in altering the enzymatic activity of butyrylcholinesterase and/or acetylcholinesterase in a subject exhibiting or predicted to exhibit cognitive disorders associated with diabetes. Subjects may be suffering from or predicted to suffer from abnormal acetylcholinesterase and/or butyrylcholinesterase activity levels or from an inability to metabolize or catabolize blood sugar normally. The method comprises administering to the subject an effective amount of MHI tartrate dispensable in discrete pharmaceutically useful dosages. MHI tartrate effectively inhibits both acetylcholinesterase and butyrylcholinesterases and additionally is highly selective for butyrylcholinesterase over acetylcholinesterase.

### CROSS-REFERENCE TO RELATED APPLICATION

This patent application claims benefit, under 35 U.S.C. § 119(e), to U.S. Provisional Patent Application No. 60/624,717, filed on Nov. 3, 2004.

### TECHNICAL FIELD

The invention relates generally to the field of biotechnology, medicine and the treatment of diabetes and its sequela and, more specifically, to a butyrylcholinesterase inhibitor and its use in the treatment and prevention of cognitive disorders associated with diabetes.

### BACKGROUND

The BCHE gene, and the activity of its gene product (BChE), the gene product being defined as the polypeptide product given by translation of the BCHE polynucleotide into its corresponding protein, is genetically linked to such diseases as vascular dementia, Alzheimer's disease (hereinafter referred to as “AD”), and diabetes. In more than 90% of Type 2 diabetes cases, pancreatic islet amyloid polypeptide is present as an insoluble refolded fibril. Such amyloidosis of islet cells is correlated with loss of islet beta cells and need for exogenous insulin therapy. (See Johnson, A. et al., Large-scale studies of the functional K variant of the butyrylcholinesterase gene in relation to Type 2 diabetes and insulin secretion, Diabetologica, 2004, 47:1437-1441). Similar amyloid fibrils are causally linked vascular dementia diseases such as AD. Amyloidosis in AD is directly correlated with cytotoxic effects. The BCHE gene, is mapped to a locus on the 3q26 chromosome, which is genetically close in proximity to a gene locus on the 3q27 chromosome linked to Type 2 diabetes. (See Vionnet, N., et al. Genomewide search for Type 2 diabetes susceptibility genes in French Whites: evidence for a novel susceptibility locus for early-onset diabetes on chromosome 3127-qter and independent replication of a Type 2 diabetes locus on chromosome 1q21-q24, Am. J. Hum. Genet., 2000, 67:1470-1480).

BChE is normally found in plasma and most tissues. BCHE exists as a single copy gene in mammals. A very common mutation of the BCHE gene is found at G1615A which is predicted to cause an alanine to threonine change in the BCHE gene product. This mutation, called the K variant, is found in 20% of Caucasians and results in a 30% reduction of BCHE activity. (See Hashim, Y. et al., Butyrylcholinesterase K variant on chromosome 3q is associated with Type II diabetes in white Caucasian subjects, Diabetologia, 2001, 44:2227-2230). Hashim, Y. et al. report “[a]n increased frequency (p=0.00079) of subjects homozygous for the BCHE K variant (AA) was observed in newly diagnosed Type 2 diabetic subjects (n=276) compared with the non-diabetic control group (n=348).” This represents a “44% increased risk of having diabetes associated with the presence of the K variant.” However, the more recent report of Johansen et al. (2004) Large-scale Studies of the Functional K Variant of the Butyrylcholinesterase Gene in Relation to Type 2 Diabetes and Insulin Secretion, Diabetologia 47:1437-1441 teaches away from an association between common mutations in the BCHE gene and type 2 diabetes.

The biological role of BCHE is not entirely understood. Aside from its role in regulating plasma acetylcholine levels, it is known that BCHE plays a role in the degradation of succinylcholine, hydrolysis of heroin and related drugs, digestion and removal of plant esters and phytotoxins, and in lipid/lipoprotein metabolism.

It has been reported that erythrocyte membrane protein glycosylation increases by 3.4 fold in diabetes (Dave, et al., Indian J Clinical Biochem., 2001, 16(1):81-88). However, insulin or sulfonylurea treatment did not reduce the extent of glycosylation. Dave, et al. also reported that serum BChE activity was low in diabetic and insulin treated diabetic groups. The diabetic state exhibited a decreased Vmax for components I and II of serum BChE. Further, in vitro incubation with insulin differentially affected the Na plus, K plus-ATPase and serum BCHE activities.

In another study, adult Long Evans rats induced into a diabetic state using streptozotocin exhibited significantly reduced BChE activity (by as much as 30-50%) in retinal tissue during the first week of hyperglycemia. (See, Sanchez-Chavez, G. et al., Effect of Streptozotocin-induced diabetes on activities of cholinesterases in the rat retina, IUBMB Life, 2000, 49:283-287). Sanchez-Chavez, G. et al. further discovered significantly decreased BChE activity (up to 50% reduction) in the rat hippocampus.

In a similar study using rats of the Charles Foster strain, it was found that serum cardiac BChE activity, including that of soluble and membrane bound forms, was increased where alloxane was used to induce the diabetic state. (See, Dave, K. R. et al., Effect of alloxan-induced diabetes on serum and cardiac butyrylcholinesterases in the rate, 2002, J. Endocrin., 175:241-250).

The invention also relates to pharmaceutical compositions comprising an effective amount of MHI tartrate, and a method for reducing the risk of AD associated with diabetes mellitus and/or treating or preventing AD using MHI tartrate.

The invention also relates to a method comprising administering to a subject an effective amount of MHI tartrate or a pharmaceutical composition of MHI tartrate, and also administering a hypoglycemic agent selected from the group consisting of sulfonylureas, meglitinides, biguanides, thiazolidinediones, alpha-glucosidase inhibitors, equivalents and mixtures thereof.

The invention relates to a method for producing a surprisingly highly soluble tartrate acid addition salt of MHI and for use of MHI tartrate in pharmaceutically acceptable compositions including excipients in the treatment of subjects.

The invention also relates to the use of MHI tartrate to treat a subject, the treatment comprising administering an effective amount of MHI tartrate or an effective amount of a pharmaceutical composition of MHI tartrate to the subject, e.g., a mammal, such as a human, thought to be in need, or predicted to be in need, of such treatment.

The invention further relates to a method of manufacturing a pharmaceutical composition comprising MHI tartrate useful, inter alia, in the treatment of diabetes mellitus and/or the risk of vascular dementia where MHI tartrate is incorporated into a form which is dispensable in discrete pharmaceutically useful dosages.

### DETAILED DESCRIPTION OF THE INVENTION

Described is the synthesis and use of an easy to manipulate and utilize, soluble salt of a potent, reversible BCHE inhibitor, MHI tartrate, for use in altering the enzymatic activity of BCHE and/or ACHE in a subject in need thereof.

MHI tartrate, synthesized as disclosed herein, is a stable salt, has the ability cross the blood brain barrier, and most especially has a uniquely high solubility allowing efficient and broad use in pharmaceutical preparations. (See, for example, U.S. Pat. Nos. 6,683,105 and 6,410,747, the contents of which are incorporated by this reference).

Carbamates are salts of carbamic acid. Carbamic acid is essentially an ester in which the carboxyl carbon is covalently linked to an amine and has the general formula of R₁- The Dave, K. R. et al. study further revealed that BCHE activity in induced diabetic rats was markedly increased in the plasma, pancreas and adipose tissues to between 33-100% above normal basal level activity. Dave, K. R. et al. cite recent literature reports showing increases in cardiac BChE activity in the diabetic rat, mouse, and human of between 22% and 270%. In Dave, K. R. et al., it is hypothesized that this increase in BCHE activity, most likely decreasing acetylcholine levels, might be a response to hypertriglyceridemia.

In addition, diabetes mellitus has been associated with an increased risk for the development of AD (Arvanitakis et al. (2004) Arch. Neurol. 61:661-666). Production and accumulation of amyloid β peptides (Aβ 1-40 and/or Aβ 1-42) are associated with AD. Hence, there is a need in the art for a medicament capable of lowering the synthesis of beta amyloid precursor protein (β APP), the precursor for Aβ, that does not produce undesirable side effects.

The references discussed herein are provided solely for the purpose of describing the field relating to the invention. Nothing herein is to be construed as an admission that the references constitute prior art or that the inventors are not entitled to antedate a disclosure by virtue of prior invention.

### SUMMARY OF THE INVENTION

The invention relates to a method of treating diabetes in a subject believed to be suffering from diabetes mellitus, comprising treating a subject with an effective amount of the tartrate acid addition salt of the compound (−)-(3aS)-3a-methyl-1,2,3,3a,8,8a-hexahydropyrrolo-[2,3-b]indol-5-yl N-4′-isopropylphenylcarbamate (herein-after referred to as “MHI tartrate”).

MHI tartrate may be used as a therapeutic agent for the purpose of attenuating the activity of butyrylcholinesterase (BChE) and acetylcholinesterase (AChE) enzymes. Provided is an efficient method for synthesis of optically pure MHI tartrate which is surprisingly highly soluble and can be used in a pharmaceutical composition which is useful, inter alia, for the prevention and/or treatment of cognitive disorders. Solubility of the MHI tartrate is between about 0.007 and about 0.013 g/ml, whereas the solubility of MHI is only about 0.0005 g/ml.

The invention also relates to pharmaceutical compositions comprising an effective amount of MHI tartrate, and a method for the treatment of diabetes mellitus and/or the risk of vascular dementia using MHI tartrate. NHCO₂—R₂, where “R” is variable and can be any chemical compound. Carbamates are generally synthesized and purified as their corresponding free base. The corresponding free base of large hetero-tricyclic carbamates, such as MHI tartrate, are generally sticky, insoluble gums. (See Yu, Q, et al., Synthesis of novel phenserine-based-selective inhibitors of butyrylcholinesterase for Alzheimer's disease, J. Med. Chem., 1999, 42(10):1855-1861).

The conversion of such carbamate compounds to their corresponding acid addition salts may have an unpredictable effect on overall solubility. For example, phenethylcymserine, exists as an insoluble crystal in its free base form; conversion of phenethylcymserine to its corresponding tartrate acid addition salt does not dramatically increase solubility, as compared to its corresponding free base. Thus, it was both surprising and fortuitous to discover that the tartrate acid addition salt of MHI is highly soluble in aqueous solution.

As used herein, the phrases “treatment of diabetes,” and “management of diabetes” are used interchangeably and do not imply the realization of a complete cure of the subject. These phrases mean reduction of the symptoms of the underlying disease and/or reduction of one or more of the underlying cellular, physiological, or biochemical indicators, causes, risks or mechanisms associated with the disease of diabetes, including reduction of such symptoms or underlying indicators, causes, risks or mechanisms to below detectable levels. “Reduced,” as used in this context, means a reduction in characteristic indicators, causes or mechanisms of the disease state or reduction in a risk of an associated disease state relative to the untreated state of the disease, including, but not limited to cellular, physiological, or biochemical indicators or risks of the diseased state or associated with the disease state.

As used herein, “effective amount” means an amount of MHI tartrate administered to the subject that is effective to improve, prevent, or treat the disease condition in the subject.

As used herein, “diabetes” refers to diseases commonly associated with a subject's inability to metabolize or catabolize blood sugar or a subject's decreased ability to regulate normal insulin concentration levels. Commonly associated diabetes sequela include diabetic ketoacidosis, hyperglycemia, hypoglycemia, abnormal carbohydrate intake, diabetic neuropathy, diabetic retinopathy, kidney dysfunction, abnormal ketone levels, hyperlipidemia, coronary artery disease, vascular dementia, amyloidosis, AD and the like.

Potential cholinesterase agents may be evaluated for potency in vitro by assaying its inhibitory effect on electric eel and human red blood cell ACHE and human plasma BCHE activity in cell-free extracts (See U.S. Pat. Nos. 6,495,700; 5,409,948; 5,171,750; 5,378,723; and 5,998,460). Studies also commonly utilize aged rat models to evaluate effectiveness of therapeutic drug leads in ameliorating cognitive disorders. For instance, the free base of MHI tartrate is potent in augmenting memory processing, as evaluated in the Stone 14-unit T-maze in aged (24-26-month-old) Fischer-344 rats. (See, Greig, N. H. et al. “Butyrylcholinesterase: Its Function and Inhibition.” (ed., Giacobini, E.) Martin Dunitz, Ltd., London, 69-90, 2003, Chapter 6: Butyrylcholinesterase: its selective inhibition and relevance to Alzheimer's disease therapy at pages 80-81).

Type 2 diabetes may be a risk factor for dementia, but the associated pathological mechanisms remain unclear. However, diabetes is increasingly associated with total dementia, AD, and vascular dementia. Individuals with both Type 2 diabetes and the APOE epsilon4 allele (encoding the protein apolioprotein E) have nearly a doubled risk for AD compared with those with either risk factor. Subjects with Type 2 diabetes and the epsilon4 allele have a higher number of hippocampal neuritic plaques and neurofibrillary tangles in the cortex and hippocampus, and they have a higher risk of cerebral amyloid angiopathy. Thus, the association between diabetes and AD is particularly strong among carriers of the APOE epsilon4 allele (Peila et al., Type 2 diabetes, APOE gene, and the risk for dementia and related pathologies: The Honolulu-Asia Aging Study, Diabetes, 2002, 51(4):1256-62). The present invention provides methods of treating diabetes, for example, neurological conditions associated with diabetes.

In accordance with the invention, MHI tartrate reduces the levels of the potentially toxic amyloid-β peptide (Aβ) and that this Aβ protein produces a progressive neurodegenerative condition leading to loss of memory, characterized by the appearance of senile plaques that are primarily composed of Aβ and neurofibrillary tangle aggregates. The Aβ is a 40- to 42-residue peptide derived from a larger protein βAPP, which is converted into the Aβ protein by proteolytic cleavage of βAPP. Aβ accumulation is one of the pathological hallmarks of cognitive impairments. Therefore, use of MHI tratrate to reduce Aβ levels provides a method of treating diabetes, such as the increased risk of AD caused by diabetes.

Compositions within the scope of the invention include compositions wherein MHI tartrate is contained in an effective amount to achieve its intended purpose. Effective concentrations may range from about 0.001 wt. % to about 1.0 wt. % MHI tartrate (wt. % is an expression of concentration meaning the percent by mass of the solute in the solution). MHI tartrate can be administered in any pharmaceutically acceptable amount, for example, in amounts ranging from about 0.001 gram to about 1 gram per kilogram of body weight. Based on the information presented herein, the determination of effective amounts is well within the skill of the ordinary practitioner in the art. In addition, the ordinary practitioner may formulate the dosage regimen as appropriate for the diabetic condition being treated. For example, the compositions of the invention may be administered orally (e.g., as a tablet or capsule) prior to carbohydrate intake and/or at times of hypoglycemia or hyperglycemia. Where MHI tartrate is administered prior to carbohydrate intake, the compound may be administered about 3 times a day.

MHI tartrate is generally used in pharmaceutical compositions containing the active ingredient with a carrier, vehicle, diluent and/or excipient in an amount of about 0.1 to 99 wt % and preferably about 25-85 wt %. Pharmaceutical compositions may be formulated using carriers, diluents and/or excipients known in the art, for example, see “Remington's Pharmaceutical Sciences,” Remington, J. P., Easton, Pa.: Mack Pub. Co., 1990. The compounds may be administered in any desired form, including, for example, parenterally, orally, injection, transdermally or by suppository using known methods. Oral delivery is the most especially preferred means of administration.

Either fluid or solid unit dosage forms can be readily prepared for oral administration. For example, MHI tartrate can be admixed with conventional ingredients such as dicalcium phosphate, magnesium aluminum silicate, magnesium stearate, calcium sulfate, starch, talc, lactose, acacia, methyl cellulose and functionally similar materials as pharmaceutical excipients or carriers. A sustained release formulation may optionally be used where appropriate or desirable. Capsules may be formulated by mixing MHI tartrate with an inert pharmaceutical diluent and inserting this mixture into a hard gelatin capsule having the appropriate size. If soft capsules are desired, then a slurry of the compound with an acceptable vegetable, light petroleum or other inert oil can be encapsulated in a gelatin capsule or similar capsules.

Suspensions, syrups and elixirs may be used for oral administration of fluid unit dosage forms. A fluid preparation including oil may be used for oil soluble forms. A vegetable oil such as corn oil, peanut oil or sunflower oil, for example, together with flavoring agents, sweeteners and any preservatives produces an acceptable fluid preparation. A surfactant may be added to water to form a syrup for fluid unit dosages. Hydro-alcoholic pharmaceutical preparations may be used having an acceptable sweetener (such as sugar, saccharin, or a biological sweetener, preferably a low carbohydrate sweetener, such as manitol or sorbitol) and a flavoring agent in the form of an elixir.

Pharmaceutical compositions for parenteral and suppository administration can also be obtained using techniques standard in the art. In an exemplary embodiment, MHI tartrate is administered as a pharmaceutical agent suitable for oral administration. In another exemplary embodiment, MHI tartrate may be injected using an appropriate vehicle such as saline.

The pharmaceutical carriers acceptable for the purposes of this invention include carriers that do not adversely affect the drug, the host, or the material comprising the drug delivery device. Suitable pharmaceutical carriers include sterile water, saline, dextrose, dextrose in water or saline condensation products of castor oil and ethylene oxide (combining about 30 to 35 moles of ethylene oxide per mole of castor oil), liquid acid, lower alkanols, oils such as corn oil, peanut oil, sesame oil and the like, with emulsifiers such as mono- or diglyceride of a fatty acid; or a phosphatide, e.g., lecithin, and the like; glycols, polyalkylene glycols, aqueous media in the presence of a suspending agent, for example, sodium carboxymethyl cellulose, sodium alginate, poly(vinylpyrrolidone), and the like, alone, or with suitable dispensing agents such as lecithin, polyoxyethylene stearate, and the like. The carrier may also contain adjuvants such as preserving agents, stabilizing agents, wetting agents, emulsifying agents and the like together with penetration enhancers and MHI tartrate.

The effective dose for mammals may vary due to such factors as age, weight, activity level or condition of the subject being treated. Typically, an effective dosage of MHI tartrate is from about 1.4 to about 1120 milligrams when administered by, for example, either oral or rectal dose from 1 to 3 times daily. This is from about 0.002 to about 50 milligrams per kilogram of the subject's weight administered per day. Preferably from about 1 to about 500 milligrams are administered orally or rectally 1 to 3 times a day for an adult human. In an exemplary embodiment, about 5-500 mg/day is administered p.o. to the subject. The required dose may be considerably less when MHI tartrate is administered parenterally. Preferably, about 0.014 to about 250 milligrams may be administered intramuscularly, one to three times per day for an adult human. In an exemplary embodiment, MHI tartrate is administered to a subject, such as a human, at a dosage of about 10 mg to about 500 mg per day.

In an exemplary embodiment, the method includes administering an effective amount of MHI tartrate or an effective amount of a pharmaceutical composition containing MHI tartrate to a subject, such as a mammal (e.g. a human), thought to be in need of such treatment. For example, a subject which may benefit from the method is a subject believed to be suffering from insulin resistance, diabetes, and/or cognitive disorders associated with diabetes.

The invention also includes a method of preparing a pharmaceutical composition useful in, among other things, treating or preventing cognitive disorders associated with diabetes, for example vascular dementia.

In another exemplary embodiment, MHI tartrate is administered in combination with insulin. For example, MHI tartrate may be administered in combination with a bolus of insulin, either an insulin injection or the action of an agent which stimulates the release of insulin. In another exemplary embodiment, MHI tartrate is administered prior to each meal to aid in efficient absorption and uptake of the drug.

While not wishing to be bound by theory, the following may help those of skill in the art to understand the mode of the invention: MHI tartrate is believed to inhibit BCHE throughout the body, causing an increase in acetylcholine concentration thus allowing normal functioning of synaptic pathways. (See, Greig, N. H. et al. at page 81). In addition, MHI tartrate reduces neurofibrillary tangle formation and decreases α-amyloid aggregation, thereby reducing the risk of developing vascular dementia, which is associated with diabetes.

In an exemplary embodiment of the invention, MHI tartrate is used to reduce the presence and/or accumulation of the β-amyloid protein found in cognitive disorders associated with diabetes.

As will be recognized by a person of ordinary skill in the art, treatment of diabetes, such as Type 2 diabetes, depends on numerous variables. This includes variations, such as, in the subject's blood glucose levels (hyperglycemia or hypoglycemia), carbohydrate intake levels, response to hypoglycemic agents, diabetic neuropathy, diabetic retinopathy, vascular dementia, kidney function, pregnancy status, ketone levels, degree of hyperlipidemia, and extent of coronary artery disease, if any.

While the invention is described in certain embodiments herein, this invention can be further modified within the spirit and scope of this disclosure. This invention is therefore intended to encompass any variations, uses, or adaptations of the invention using the invention's general principles. Further, this invention includes such variations on the present disclosure as come within known or customary practice in the art to which this invention pertains and which fall within the limits of the appended claims.

### EXAMPLE I

### Synthesis of the Free Base Precursor of MHI Tartrate

MHI tartrate is synthesized from the commercially available alkaloid, physostigmine. (See, Zhu, Xiaoxiang, et al. A practical conversion of natural physostigmine into the potent butyrylcholinesterase inhibitor N¹, N⁸-bisnorcymserine, 2000, Tet. Lett., 4861-4864). (−)-Physostigmine is treated with sodium n-butoxide in n-butanol to give eseroline. (−)-Eseroline is then purified and isolated as its fumarate salt, and, thereafter, converted into N¹-benzylnoresermethole, according to procedures known in the art.

To a solution of N¹-benzylnoresermethole (1.54 g, 4.99 mmol) in dichloromethane (75 mL) is added NaHCO₃ (1 g). This mixture is stirred vigorously and cooled in an ice bath. Pyridinium dichromate (3.76 g, 9.99 mmol) is then added, and the mixture is stirred for two hours. The reaction mixture is filtered and the resulting solid is washed with dichloromethane (50 mL). The combined dichloromethane solution is washed with water three times (50 μL each wash), dried over anhydrous Na₂SO₄ and concentrated under vacuum.

The remaining residue is purified by silica gel column chromatography using petroleum ether:ethyl acetate (10:1 and 10:2) as the eluent to obtain starting material N-benzylnoresermethole (0.15 g) and the N⁸-formyl derivative of N¹-benzylnoresermethole (0.65 g, 45%). A further elution with petroleum ether:ethyl acetate (4:1) yields a more polar component corresponding to N¹-benzylnoresermethole wherein the N⁸-methyl group is removed (0.235 g, 19%).

The N⁸-formyl derivative of N¹-benzylnoresermethole is reacted with BBr₃ in dichloromethane for one hour at room temperature after which sufficient methanol is added to destroy any excess BBr₃. The remaining residue after evaporation is purified by flash chromatography to afford N¹-benzylbisnorseroline (77%). This step provides a valuable “one pot” reaction in which both O-demethylation and N-deformylation occur simultaneously.

The last silica gel fraction, containing the N¹-benzylnoresermethole wherein the N⁸-methyl group is removed, is reduced with sodium borohydride in tetrahydrofuran to afford N-benzylbisnorseroline (85%). N¹-Benzylbisnorseroline is incubated with 4-isopropylphenyl isocyanate yielding N¹-benzyl-N⁸-norcymserine. According to published methods, conversion to N¹,N⁸-bisnorcymserine (MHI carbamate) is achieved through catalytic hydrogenation using Pd(OH)₂/C as a catalyst and iso-propanol as the solvent to affect N-debenzylation of N-benzyl-N⁸-norcymserine. Overall yield is 20%.

### EXAMPLE II

### Physical and Biochemical Characterization of MHI Carbamate

Pertinent physical properties of MHI carbamate are as follows: [a]_{D}²⁰-71.1° (c=0.3, CHCl₃); ¹H NMR (CDCl₃) δ7.29 (d, J=8.5 Hz, 2H, C2′-H and C6′-H), 7.10 (d, J=8.5 Hz, 2H, C3′-H and C5′-H), 6.80 (m, 2H, C4-H and C6-H), 6.55 (d, J=8.5 Hz, C7-H), 5.20 (s, 1H, C8a-H), 2.90 (m, 1H, Ph-CH<), 2.80 (m, 2H, C2-H₂), 2.13 (m, 2H, C3-H₂), 1.45 (s, 3H, C3a-CH₃), 1.18 (d, J=7.0 Hz, >CMe₂); EI-MS m/z (relative intensity) 190 (MH⁺—ArNHCO, 98), 174 (10), 160 (70), 146 (100), 133 (11), 117 (15), 103 (5.0), 91 (14); HR-MS m/z calcd for C₂₁H₂₅N₃O₂ 351.1948, found 351.1941. (See, Yu, Qian-sheng, et al. Synthesis of Novel Phenserine-Based-Selective Inhibitors of Butyrylcholinesterase for Alzheimer's Disease, 1999, J. Med. Chem., 42: 1855-1861, at page 1859).

Biological activity assays reveal that MHI carbamate derived using the synthetic pathway detailed in EXAMPLE I gives IC₅₀ values (the concentration required to inhibit 50% of enzyme activity assayed) for BChE and AChE of 1.0±0.1 nM and 110±15 nM, respectively. (See, Greig, N. H. et al. at page 78). These biological activity assays use human BChE and AChE freshly prepared from human plasma and erythrocytes, respectively.

### EXAMPLE III

### Synthesis of MHI Tartrate

Under an argon atmosphere, a solution of tartaric acid in a mixture of anhydrous ethanol and deionized water is added slowly to a slurry of MHI carbamate, also in a mixture of anhydrous ethanol and deionized water, in a 1:1 mole ratio (carbamate:tartrate). After about two-thirds of the tartrate solution is added, the reaction is seeded with MHI tartrate. This mixture is stirred for several hours at room temperature. Acetone is then added and the mixture stirred for several more hours. The precipitate is filtered via Büchner funnel and collected on filter paper. The white crystalline solid is washed with acetone and dried to yield MHI tartrate.

### EXAMPLE IV

### Physical and Biochemical Characterization of MHI Tartrate

Pertinent physical properties of MHI carbamate are as follows: [a]_{D}²⁰-41.67° (c=0.1, EtOH); ¹H NMR (CDCl₃) δ7.29 (d, J=8.5 Hz, 2H, C2′-H and C6′-H), 7.10 (d, J=8.5 Hz, 2H, C3′-H and C5′-H), 6.80 (m, 2H, C4-H and C6-H), 6.55 (d, J=8.5 Hz, C7-H), 5.20 (s, 1H, C8a-H), 2.90 (m, 1H, Ph-CH<), 2.80 (m, 2H, C2-H₂), 2.13 (m, 2H, C3-H₂), 1.45 (s, 3H, C3a-CH₃), 1.18 (d, J=7.0 Hz, >CMe₂); EI-MS m/z (relative intensity) 190 (MH⁺—ArNHCO, 98), 174 (10), 160 (70), 146 (100), 133 (11), 117 (15), 103 (5.0), 91 (14); HR-MS m/z calcd for C₂₁H₂₅N₃O₂ 351.1948, found 351.1941; Mp. 187-188° C.

Biological activity assays reveal that MHI tartrate derived using the synthetic pathway detailed in EXAMPLE III gives IC₅₀ values (the concentration required to inhibit 50% of enzyme activity assayed) for BChE and AChE of 1.0±0.1 nM and 110±15 nM, respectively. These biological activity assays use human BChE and AChE freshly prepared from human plasma and erythrocytes, respectively.

### EXAMPLE V

### Solubility of MHI Tartrate Compared to Acid Addition Salts of Similar Carbamates

| | | | Solubility |
|---|---|---|---|
| | | | (g per 100 ml |
| | | | Saturated H₂O |
| | | | solution at room |
| Compound | Mp. | [α]²⁰D | temperature) |
| MHI (Base) | — (gum) | −71.1° | 0.05 g |
| | | (c = 0.3, CHCl₃) | |
| MHI | 187-188° C. | −41.7° | 1 g |
| Tartrate | | (c = 0.1, EtOH) | |

### EXAMPLE VI

### Formulation of Pharmaceutical Compositions Containing MHI Tartrate

Individual oral dosage forms, i.e., capsules, pills, tablets, and the like, are obtained by admixing, for example, 1.4 to 1120 mg of the MHI tartrate of EXAMPLE III with a pharmaceutically acceptable, inert diluent such as talc and forming said mixture into an appropriately sized tablet, pill, or capsule. Large-scale production of such individual dosage forms is accomplished through admixing, under strictly controlled environmental conditions, some multiple of the recommended MHI tartrate dosage, for example, 10,000 times 1.4 to 1120 mg, with a corresponding multiple amount of pharmaceutically acceptable, inert diluent such as talc to enable formation of 10,000 such individual dosages.

Likewise, a gel capsule is derived by admixing 1.4 to 1120 mg of MHI tartrate with a pharmaceutically acceptable and inert oil, such as corn oil. This mixture is then encapsulated within a pharmaceutically acceptable, appropriately sized, inert container such as a gelatin capsule.

### EXAMPLE VII

### Treatment of Subjects Exhibiting Cognitive Disorders Associated With Diabetes

A subject exhibiting a cognitive disorder associated with diabetes, for instance aged (21-22 month old) Fisher-344 rats are given intraperitoneal injections of a pharmacological solution containing an effective amount of MHI tartrate dissolved in isotonic saline. (See U.S. Pat. No. 6,683,105). MHI tartrate is administered in dosages in the range of 0.5 to 1.0 mg per kg of subject body weight. This dosage is administered daily, for example, 1-3 times per day. Effectiveness of MHI tartrate is measured by testing cognitive ability before and after administration. For instance, rats may be tested using a “Stone maze” in which a maze constructed of translucent plastic containing a grid floor wired for scrambled foot shocks surrounded by opaque gray walls to minimize effects of non-MHI tartrate related variables. Rats are given 10 seconds in which to navigate the maze. Treatment with MHI tartrate is meant to increase cognitive ability, thereby allowing the subject to navigate the maze more quickly after each daily administration of MHI tartrate. Similar cognitive tests, for instance timed, objective memorization tests, may be used on other subjects, such as humans.

All references, including publications, patents, and patent applications, cited herein are hereby incorporated by reference to the same extent as if each reference were individually and specifically indicated to be incorporated by reference and were set forth in its entirety herein.

What is claimed is:

1. A method of treating or preventing cognitive disorders associated with diabetes in a subject, the method comprising administering to the subject a pharmaceutical composition comprising:
a pharmaceutically effective amount of a tartrate salt of a compound having the formula of (−)-(3aS)-3a-methyl-1,2,3,3a,8,8a-hexahydropyrrolo-[2,3-b]indol-5-yl N-4′-isopropylphenyl-carbamate; and
a pharmaceutically acceptable excipient.

2. The method according to claim 1 wherein the tartrate salt of the compound is incorporated into the pharmaceutical composition as a crystalline solid.

3. The method according to claim 2 wherein:
the crystalline solid has a melting point of between 187 and 188 degrees Celcius.

4. The method according to claim 1, wherein:
the tartrate salt of the compound has an aqueous solubility of between about 0.007 g/mL and about 0.013 g/mL.

5. The method according to claim 1, further comprising administering to the subject the pharmaceutical composition of claim 1 one to three times daily.

6. The method according to claim 1, further comprising administering to the subject the pharmaceutical composition more than one hour prior to a meal.

7. The method according to claim 6, further comprising coadministering a hypoglycemic agent selected from the group consisting of a sulfonylurea, a meglitinide, a biguanide, a thiazolidinedione, an alpha-glucosidase inhibitor, insulin and mixtures thereof to the subject.

8. A tartrate salt of a compound having the formula of (−)-(3aS)-3a-methyl-1,2,3,3a,8,8a-hexahydropyrrolo-[2,3-b]indol-5-yl N-4′-isopropylphenylcarbamate and a pharmaceutically acceptable excipient.

9. A method of treating or preventing a diabetic complication, the method comprising:
administering an effective amount of the tartrate salt of a compound having the formula of claim 8 to a subject having diabetes.

10. The method according to claim 9, wherein:
the tartrate salt of the compound is a crystalline solid having a melting point of between 187 and 188 degrees Celcius.

11. The method of treating or preventing a diabetic complication, the method comprising:
administering an effective amount of the tartrate salt of a compound having the formula of claim 8 to a subject having diabetes.
